(19) United States
(12) Patent Application Publication
(10) Pub. No.: US 2025/0114238 A1
(43) Pub. Date: Apr. 10, 2025

(54) APPARATUS, METHOD, AND SYSTEM FOR IMPROVING NASAL BREATHING BY REDUCING MOUTH BREATHING

(71) Applicant: Oscar A. TAMEZ, Georgetown, TX (US)

(72) Inventor: Oscar A. TAMEZ, Georgetown, TX (US)

(21) Appl. No.: 18/907,763

(22) Filed: Oct. 7, 2024

Related U.S. Application Data
(60) Provisional application No. 63/588,007, filed on Oct. 5, 2023.

(51) Int. Cl. A61F 5/56

(52) U.S. Cl. CPC A61F 5/566

(57) ABSTRACT

A mouth appliance for improving nasal breathing by reducing mouth breathing. The mouth appliance can include a sealing arrangement having an upper portion attachable to upper teeth of a user and a lower portion attachable to lower teeth of the user, wherein the upper portion is couplable to the lower portion. When the upper portion is coupled to the lower portion, a lower jaw of the user is maintained in a closed position with respect to an upper jaw of the user. The upper portion can include a magnet and the lower portion can include a complementary magnet.

### CROSS-REFERENCE TO RELATED APPLICATIONS

This application claims priority to U.S. Provisional Application No. 63/588,007, filed Oct. 5, 2023, the entire contents of which are incorporated by reference herein.

### BACKGROUND

Mouth breathing is a leading cause of dental ailments and a significant cause of a wide range of other medical issues, negatively affecting craniofacial development, sleep quality, neurological function, and overall well-being. Continuous nasal breathing is important for resolving the health risks associated with mouth breathing. Mouth breathing disrupts the exchange and decreases oxygen absorption; this can lead to over breathing because one needs to breathe more frequently in order to replenish the oxygen that is being lost. Breathing through the nose also filters air and removes 98-99% of allergens, bacteria, and viruses from the air. Nasal breathing keeps air in the lungs longer than mouth breathing, and provides up to 20% more oxygen in the bloodstream at any given time. Nasal breathing also warms the air by as much as 40 degrees before it gets to the lungs, which is especially important in cold weather. Additionally, optimal exercise tolerance is dependent on nasal breathing.

The impact of mouth breathing on craniofacial growth is particularly significant in children. Mouth breathing can lead to issues such as craniofacial crowding, tongue thrusting, palate narrowing, large anterior face height, forward head posture, and impaired development of the maxilla and mandible. These craniofacial abnormalities contribute directly and indirectly to various health problems, including sleep-related breathing disorders (SRBDs), abnormal skeletal development, and orthodontic issues, such as malocclusion, teeth crowding, crossbite, and open bite deformities. Additionally, the small, narrow craniofacial structure associated with mouth breathing further increases the occurrence of mouth breathing and narrowing of the posterior airway space (PAS). This reduction in the size of the upper airway sets the stage for SRBDs, including childhood and adult sleep apnea.

During sleep, mouth breathing actively disrupts sleep patterns and contributes to the development of SRBDs, such as Upper Airway Resistance Syndrome and Obstructive Sleep Apnea. Mouth breathing causes narrowing of the PAS (Posterior Airway Space) by causing the base of tongue to fall back and obstruct the oropharynx. Disrupted sleep patterns are linked to various diseases, including hypertension, heart disease, cancer, ADHD, anxiety, Alzheimer's disease, and fatalities resulting from fatigue-induced errors. The stress caused by disrupted sleep also triggers neurologic dysfunction, leading to imbalances in the sympathetic and parasympathetic drive.

Further, mouth breathing can directly and indirectly contribute to temporomandibular disorders and facial pain. The increased sympathetic drive resulting from SRBDs can result in open bite deformities from tongue thrusting, dental wear from bruxism (clenching and grinding), fracturing of teeth and crowns, and receding gum lines. Bruxism leads to wear, inflammation, and pain in the temporomandibular joint, as well as jaw dislocation and an increase in the severity of malocclusion.

In addition to sleep and craniofacial abnormalities, mouth breathing leads to various oral health problems, including dry mouth, gingivitis, gingival hypertrophy, receding gums, cavities, plaque, halitosis, drooling, and chapped lips. The constant exposure of oral tissues to air disrupts natural lubrication, bacterial dysbiosis, and cleansing mechanisms, contributing to these issues. Mouth breathing can also exacerbate Gastroesophageal Reflux Disease (GERD), leading to tooth erosion due to exposure to stomach acid. Additionally, mouth breathing is a risk factor of chronic or recurring thrush and ulcers.

It is necessary to intervene early in the etiological factors of mouth breathing to prevent its development or worsening. Proper nasal breathing, day and night, leads to proper tongue position, maximum palate and mandibular arch dimensions, proper alignment of teeth, optimal oxygenation, and deeper sleep with resultant growth hormone secretion at night

Currently, there is a lack of comprehensive solutions to minimize mouth breathing while optimizing nasal breathing and all of its benefits. Although there are appliances available for teeth protection or other dental ailments, these appliances do not specifically address the underlying mouth breathing or promote optimal breathing. Existing mouth appliances that address mouth breathing focus on advancing the jaw forward to open the PAS but often cause shifting in the wearer's teeth, restrict the wearer's ability to speak and drink, and/or can only be worn at night. Furthermore, current appliances are ineffective in preventing the mouth from opening, rendering them ineffective for optimally promoting nasal breathing and reducing mouth breathing, or combinations thereof.

### SUMMARY

Exemplary embodiments described herein generally relate to a mouth appliance and a method to improve breathing, oxygenation, and sleeping, and to the mouth appliance and a sealing arrangement to prevent the jaw from non-purposeful opening, eliminate tension on the teeth, and allow for easy vertical and horizontal movements of the jaw without interfering with comfort, breathing, drinking, or talking.

The mouth appliance can include a sealing arrangement having an upper portion attachable to upper teeth of a user and a lower portion attachable to lower teeth of the user, wherein the upper portion is couplable to the lower portion. When the upper portion is coupled to the lower portion, a lower jaw of the user can be maintained in a closed position with respect to an upper jaw of the user. The upper portion can include a magnet and the lower portion can include a complementary magnet.

### BRIEF DESCRIPTION OF THE FIGURES

Advantages of embodiments will be apparent from the following detailed description of the exemplary embodiments thereof, which description should be considered in conjunction with the accompanying drawings in which like numerals indicate like elements, in which:

FIG. 1A is a side view of an exemplary embodiment of a mouth appliance.

FIG. 1B is a plan view of the exemplary embodiment of the mouth appliance.

FIG. 1C is a plan view of the exemplary embodiment of the mouth appliance.

FIG. 2 is a side view of another exemplary embodiment of a mouth appliance.

FIG. 3 is a side view of another exemplary embodiment of a mouth appliance.

FIG. 4 is a side view of another exemplary embodiment of a mouth appliance.

FIG. 5A is a side view of another exemplary embodiment of a mouth appliance.

FIG. 5B is a rear diagrammatic view of the exemplary embodiment of the mouth appliance in a disengaged position.

FIG. 5C is a rear diagrammatic view of the exemplary embodiment of the mouth appliance in an engaged position.

FIG. 6A is a rear diagrammatic view of another exemplary embodiment of a mouth appliance in a disengaged position.

FIG. 6B is a rear diagrammatic view of the exemplary embodiment of the mouth appliance in an engaged position.

FIG. 7A is a rear diagrammatic view of another exemplary embodiment of a mouth appliance in a disengaged position.

FIG. 7B is a rear diagrammatic view of the exemplary embodiment of the mouth appliance in an engaged position.

FIG. 8A is a rear diagrammatic view of another exemplary embodiment of a mouth appliance in a disengaged position. FIG. 8B is a rear diagrammatic view of the exemplary embodiment of the mouth appliance in an engaged position.

FIG. 9 is a plan view of another exemplary embodiment of a mouth appliance.

FIG. 10 is a side view of another exemplary embodiment of a mouth appliance.

### DETAILED DESCRIPTION

Aspects of embodiments are provided in the following description and related drawings directed to specific embodiments. Alternate embodiments may be devised without departing from the spirit or the scope of the embodiments shown and described herein. Additionally, well-known elements of exemplary embodiments will not be described in detail or will be omitted so as not to obscure the relevant details. Further, to facilitate an understanding of the description discussion of several terms used herein follows.

As used herein, the word “exemplary” means “serving as an example, instance or illustration.” The embodiments described herein are not limiting, but rather are exemplary only. It should be understood that the described embodiments are not necessarily to be construed as preferred or advantageous over other embodiments. Moreover, the terms “embodiments of the invention”, “embodiments” or “invention” do not require that all embodiments of the invention include the discussed feature, advantage or mode of operation.

According to an exemplary embodiment, and referring generally to the Figures, various exemplary implementations of a mouth appliance may be provided. According to exemplary embodiments, the mouth appliance may be designed to improve breathing, sleeping, and/or teeth alignment and/or reduce bruxism, teeth grinding, and tongue thrusting. In particular, mouth appliances may be designed to promote nasal breathing. The mouth appliance can include an upper mouthpiece, a lower mouthpiece, and a sealing arrangement. The mouth appliance can be a bruxism appliance, another sports mouth appliance, orthodontic aligner or retainer, sleep apnea or snoring device, or the like.

According to exemplary embodiments, the mouth appliance may be provided in various formats. For example, the mouth appliance may be provided in the form of a sports mouth guard, may be moldable or conformed to the teeth of the wearer, and/or may be configured for wear during athletics. As another example, the mouth appliance may be provided in the form of a device for reducing bruxism or teeth grinding, a device for protecting the teeth, as an orthodontic appliance for straightening, aligning, and/or retaining teeth, as a mouth appliance to prevent thumb sucking or tongue thrusting, or as an appliance for snoring/sleep apnea. It may also be provided so as to be disposed in the mouth without interfering with comfort, breathing, drinking, or talking. In yet other exemplary embodiments, the upper and lower mouth pieces may conform solely to the molar region of a patient's mouth and may be connected by a generally U-shaped horizontal portion, such as in the form of a dental aligner. In still other embodiments, the mouth appliance may be configured as a dental aligner to incrementally move the teeth as normally associated with orthodontic treatment or to prevent movement of teeth or to protect the teeth from bruxism. In some exemplary embodiments, a frame of the mouth pieces may be made with medically approved, orthodontic grade, flexible, moldable materials such as plastic, polyethylene-polyvinyl acetate copolymer, silastic, silicone, acrylic, latex or any other soft material which has a standard FDA approval and is BPA (bisphenol) free. In yet other embodiments, attachment devices, such as brackets, hooks or wires, for the mouth appliance may be provided for attaching to teeth and/or braces. In such embodiments, separate attachment and engagement elements, including magnets, can be configured to couple to the attachment devices.

The sealing arrangement may be located on the upper and lower mouthpiece of the mouth appliance and disposed on the buccal side of the teeth and/or on lingual, anterior, or interdental locations. The sealing arrangement may be permanently or removably coupled to the upper and lower mouthpiece. In some embodiments, a bonding agent may be used to attach or embed the sealing arrangement to the mouthpiece or to the surface of the teeth. The bonding agent may include medically approved, orthodontic, or prosthodontic grade resins, which may have a standard FDA approval and be BPA (bisphenol) free. The sealing arrangement may include magnetic elements to create a seal between the upper and lower mouthpiece as to prevent the jaw from non-purposeful opening, reduce or eliminate tension on the teeth, and allow for easy vertical and horizontal movements of the jaw.

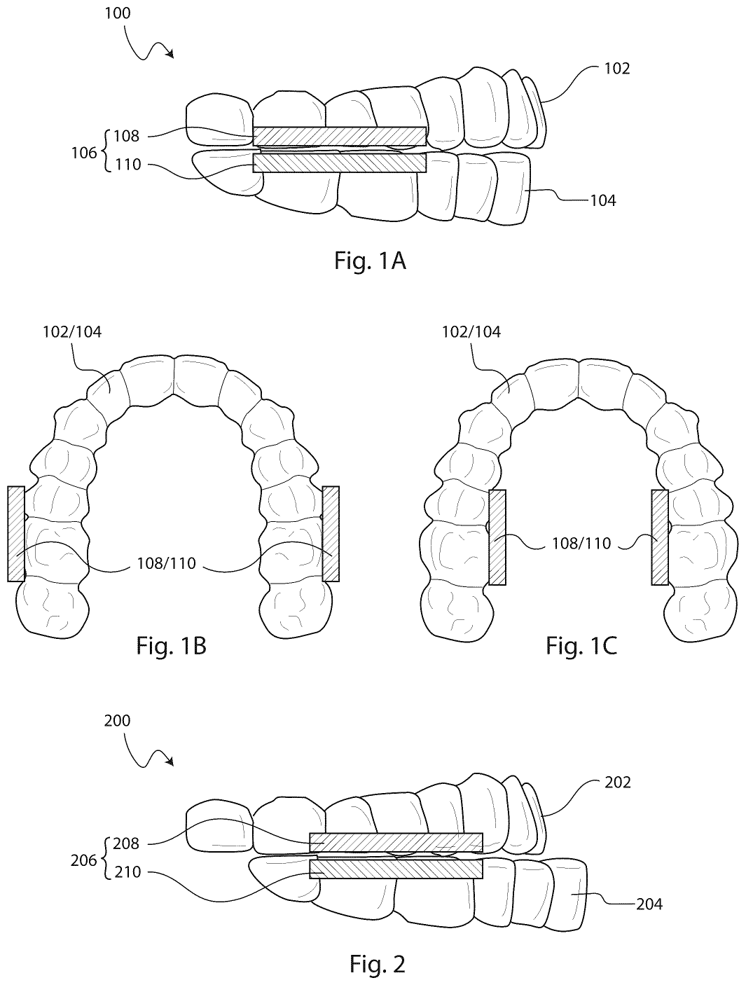

In some exemplary embodiments, the sealing arrangement may include magnetic elements located on the upper and lower mouthpieces. FIG. 1A shows a side view of an exemplary mouth appliance 100 provided as a teeth retainer/aligner. The mouth appliance can include an upper mouthpiece 102, a lower mouthpiece 104, and a sealing arrangement 106 having a magnetic element 108 and a complementary magnetic element 110.

FIGS. 1B-1C show plan views of an exemplary lower mouthpiece 102 or upper mouthpiece 104 of the mouth appliance 100. FIG. 1B shows the magnetic elements 108/110 having a rectangular shape and extending along the buccal sides of the mouth pieces. Positioning the magnets on the buccal side of the mouthpieces may be less constrictive to the user and avoid interference with the tongue. In other exemplary embodiments, the sealing arrangement may be disposed on the lingual or labial sides, as shown in FIG. 1C. In yet other embodiments, the magnetic elements of the sealing arrangement may be positioned interdentally.

The magnetic elements may vary in composition, strength, size, placement, and number. In an exemplary embodiment, the magnetic elements may include neodymium plated with a protective coating, such as nickel (Ni-Cu-Ni), gold (Ni-Cu-Ni-Au), epoxy (Ni-Cu-BE), Teflon, or plastic. Additionally, the magnetic elements may further be coated with medically approved materials, such as waterproof plastic, epoxy, rubber, stainless steel, gold, platinum, or titanium. In an exemplary embodiment, the magnetic elements may feature grade N42 magnets, which may have a maximum energy product of about 42 MGOe. Further, the magnetic elements may have a rectangular, square, circular, other shape, as desired. Exemplary dimensions for the magnetic elements may be 1″×⅛″×⅛″; however, it should be appreciated that various sizes that enable the mouth appliance to function as described herein may be used. Additionally, in embodiments with interdental positioning of the magnetic elements, it may be desired to use thinner magnets so as to reduce temporal mandibular joint (TMJ) displacement from increasing interdental height. Further, in some embodiments, the mouth appliance may have a built-in sliding device that can allow for jaw advancement or mandibular adjustment.

Exemplary magnetic elements configured to be placed on an occlusal or interdental surface without affecting vertical height may be substantially flat and circular, so as to avoid displacement of the temporal mandibular joint (TMJ) may not be displaced. Exemplary dimensions for such magnets may be 5 mm ×5 mm ×2 mm. Such circular magnets can be used in other locations, such as buccal, lingual anterior, etc. In yet other embodiments, any mechanical structure which creates a seal between the upper and lower mouth pieces while allowing for vertical and horizontal movements of the jaw may be contemplated and provided as desired.

FIG. 2 illustrates advancement of the mandible by an exemplary embodiment of a mouth appliance 200. The magnets 208/210 of sealing arrangement 206 may be positioned on the upper/lower mouthpieces 202/204 so as to cause the lower teeth to be advanced several millimeters with respect to the upper teeth such that the lower jaw remains forwardly of the lower jaw and the jaws remain substantially closed during sleep. Such a configuration allows for maintaining an advanced lower jaw position and preventing displacement, while at the same time preventing the mouth from opening and thereby causing the PAS to narrow anatomically as well as allowing the tongue to fall posteriorly.

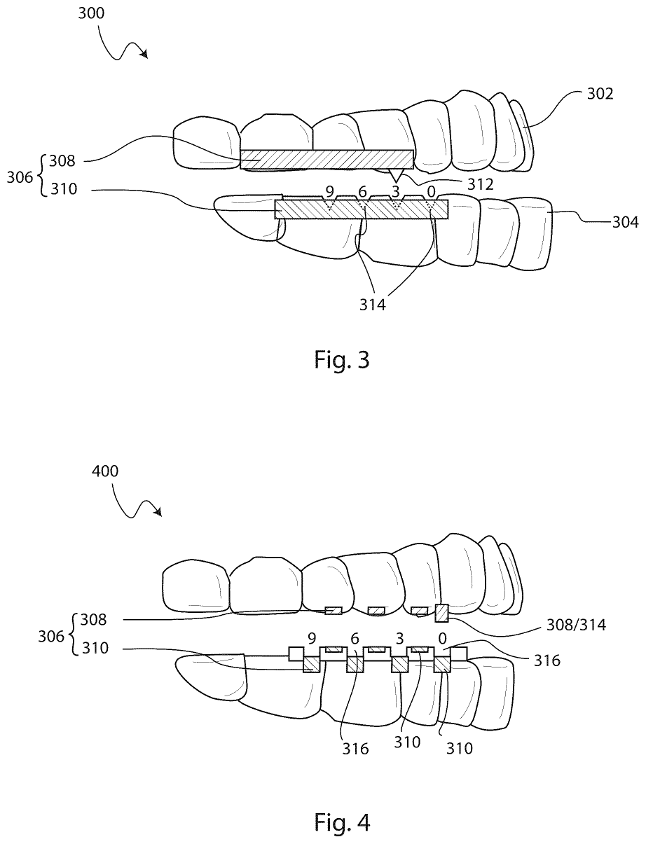

FIG. 3 shows another exemplary embodiment of a mouth appliance 300, wherein the mandibular advancement may be varied. In the exemplary embodiment, the sealing arrangement may include magnetic elements 308/310. The magnetic elements may be adjusted for mandibular advancement. For example, adjustments may be made at intervals of about 3 mm. The adjustment may be effected by the relative locations of the magnetic elements on the upper and lower mouthpieces or by physically interlocking components. To achieve a desired advancement, the user can open their mouth and move the lower jaw forward or backward, as desired, so as engage the desired amount of advancement. Alternatively, the advancement may be set to the desired distance when the mouth appliance is outside the mouth, the mouth appliance then being placed in the mouth with the advancement already set.

In the embodiment of FIG. 3, when magnetic elements 308/310 are located laterally or buccally, one of the upper or lower mouthpieces may include a triangular or square male peg 312 that fits into one of several complementary receptacles 314 in the other of the upper and lower mouthpieces. For example, the peg can be set into a neutral, or “0”, position, which position can then be advanced, for example, in intervals of 3 mm, to a 3 mm position, 6 mm position, or 9 mm position. Other intervals may be contemplated and provided as desired. The magnetic elements may function to maintain the mouth appliance in an active or closed position, while the pegs and receptacles can further facilitate preventing disengagement or movement from a desired position.

FIG. 4 shows an exemplary embodiment of a mouth appliance 400, where one or more magnetic elements 408/410 are positioned interdentally, rather than laterally or buccally. In the exemplary embodiment, one of the upper or lower mouthpieces 402/404 may include a triangular or square male peg 412 that fits into one of several complementary receptacles 414 in the other of the upper and lower mouthpieces. For example, the peg 412 can be set into a neutral, or “0”, position, which position can then be advanced, for example, in intervals of 3 mm, to a 3 mm position, 6 mm position, or 9 mm position. Other intervals may be contemplated and provided as desired. The magnetic elements may function to maintain the mouth appliance in an active or closed position, while the pegs and receptacles can further facilitate preventing disengagement or movement from a desired position.

In some exemplary embodiments, a mouth appliance may include an attachment device for removably joining the sealing arrangement. In other embodiments, a mouth appliance may include an attachment device so as to activate or deactivate a sealing arrangement. The attachment device may be made from a variety of materials and may change in shape, stiffness, or orientation to implement such joining. The attachment device may feature a variety of designs, including hinges, hooks, clasps, bands, brackets, snaps, springs, levers, rods, tubes, coils, pockets, indents, and/or other protrusions. For example, the attachment device may be sized to allow for the sealing arrangement to connect to an upper or lower mouthpiece. The attachment device may include multiple mechanisms and may be attached or attachable to the upper and lower mouthpieces, the sealing arrangement, or the teeth.

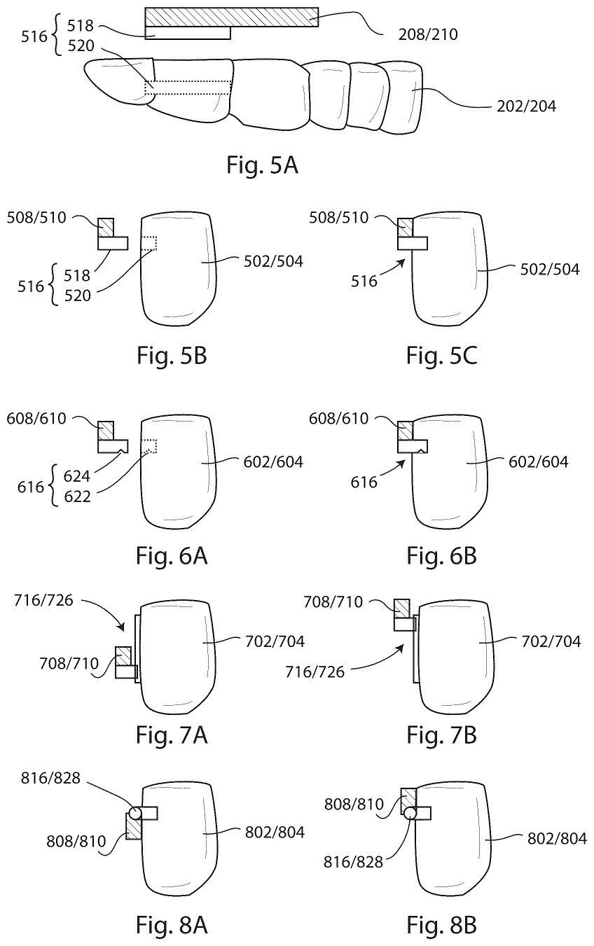

Referring to FIGS. 5A-5C, in an exemplary embodiment of a mouth appliance 500, a mouth appliance may have an attachment device 516 that includes a pocket 518 which receives a complementary member 520 of a corresponding sealing arrangement 106. FIG. 5A shows an exemplary side view of a lower mouthpiece 504 of mouth appliance 500, showing schematically an exemplary positioning of the pocket 518 and complementary member 520. FIG. 5B shows the exemplary sealing arrangement 506 detached from mouthpiece 504, while FIG. 5C shows the exemplary sealing arrangement attached to the mouthpiece. It should be appreciated that while only the lower mouthpiece is shown, the upper mouthpiece can be adapted in the same way.

In yet another exemplary embodiment of a mouth appliance 600, as shown in FIGS. 6A-6B, an attachment device 616 may feature an embedded projection 622 and complementary depression 624.

Referring to FIGS. 7A-7B, in an exemplary embodiment of a mouth appliance 700, a mouth appliance may have an attachment device 716 that may include a mechanism for shifting the sealing arrangement 706, which may allow for activation or deactivation of the sealing arrangement 706. In an exemplary embodiment, the attachment device may include a sliding mechanism 726 such that the sealing arrangement may slide vertically on the attachment device. In an exemplary embodiment, the sliding mechanism may feature a rail.

In yet another exemplary embodiment of a mouth appliance 800, as shown in FIGS. 8A-8B, an attachment device 816 may include a hinge mechanism 828. In some such embodiments, a hinge may be secured to the buccal side of the mouth appliance in between the mouth appliance plastic and the teeth for a snug or desired fit, such that the magnetic element of the sealing arrangement may be pivotable.

In yet further embodiments, the attachment device may include a locking mechanism so as to removably anchor the sealing arrangement in an active position with respect to the mouthpiece. The locking mechanism may be implemented in various manners, for example, including hinges, hooks, clasps, snaps, springs, coils, buttons, slides, and latches. In an exemplary embodiment, a locking mechanism may include a coil with a hinge such that the locking mechanism may be activated when the hinge is lowered. In another exemplary embodiment, a locking mechanism may include a push button which may activate and deactivate a coil and hinge mechanism. In still other embodiments, a locking mechanism may include a latch with a push button. In yet further embodiments, a magnetic element may be rotated, stacked, and/or slid onto a plastic element to activate and deactivate the magnetic component.

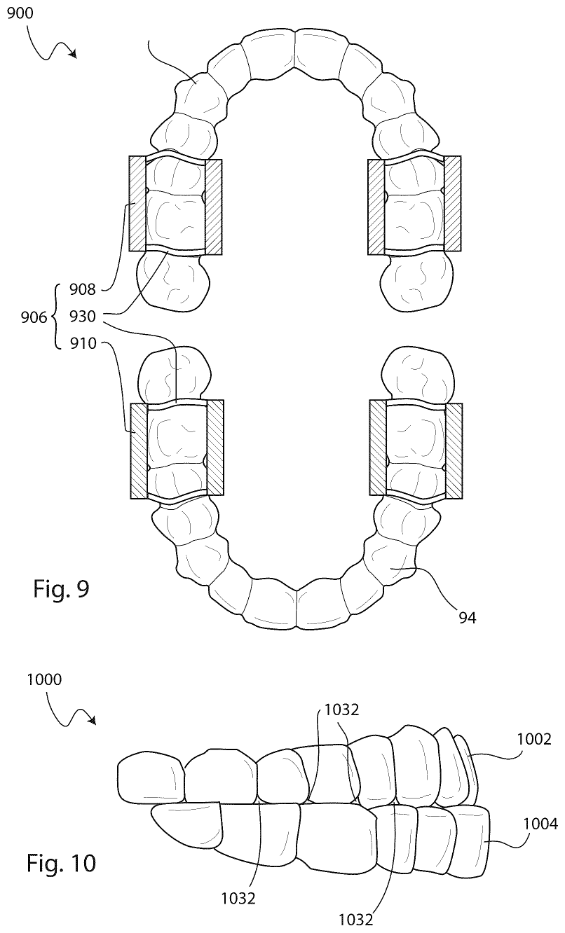

In yet another exemplary embodiment, as shown in FIG. 9, a sealing arrangement may be designed such that upper and lower mouth pieces are not needed. In the exemplary embodiment 900, a sealing arrangement 906 may be fitted onto the upper 92 and lower teeth 94, and may include magnetic elements 908/910 as to prevent the jaw from non-purposeful opening, eliminate any tension on the teeth, and allow for easy vertical and horizontal movements of the jaw without the need for upper and lower mouth pieces. The sealing arrangement 906 may include a flexible material 930 linking the magnetic elements 908/910 on buccal and lingual sides of the teeth, which may allow the magnetic elements to conform to both the lingual and buccal sides of the teeth to form a snug or otherwise desired fit.

FIG. 10 shows yet another embodiment of a mouth appliance 1000. Mouth appliance 1000 may be a low profile, one-piece appliance having upper mouthpiece 1002 and lower mouthpiece 1004 sealed together. Small holes 1032 may be provided interdentally so as to allow for the passage of air. The embodiment of mouth appliance 1000 may not include a magnetic element, and can be placed, worn, or used during exercise or sleep as one unit and can be removed, as desired.

The foregoing description and accompanying figures illustrate the principles, embodiments and modes of operation. However, the scope should not be construed as being limited to the particular embodiments above. Additional variations of the embodiments will be appreciated by those skilled in the art (for example, features associated with certain configurations of the embodiments may instead be associated with any other configurations, as desired).

Therefore, the above-described embodiments should be regarded as illustrative rather than restrictive. Accordingly, it should be appreciated that variations to those embodiments can be made by those skilled in the art without departing from the scope of the embodiments.

What is claimed is:

1. A mouth appliance for improving nasal breathing by reducing mouth breathing, comprising:
a sealing arrangement having an upper portion attachable to upper teeth of a user and a lower portion attachable to lower teeth of the user;
wherein the upper portion is couplable to the lower portion; and
wherein, when the upper portion is coupled to the lower portion, a lower jaw of the user is maintained in a closed position with respect to an upper jaw of the user.

2. The mouth appliance of claim 1, wherein the upper portion includes a magnet and the lower portion includes a complementary magnet.

3. The mouth appliance of claim 1, wherein the upper portion directly couplable to the upper teeth of the user, and the lower portion of the sealing arrangement is directly couplable to the lower teeth of the user.

4. The mouth appliance of claim 1, wherein the upper portion and the lower portion are arranged such that, when the upper portion is coupled to the lower portion, the lower jaw is advanced with respect to the lower jaw.

5. The mouth appliance of claim 1, wherein the upper portion of the sealing arrangement is coupled an upper mouthpiece that is configured to couple to the upper teeth of the user, and the lower portion of the sealing arrangement is coupled to a lower mouthpiece that is configured to couple to the lower teeth of the user.

6. The mouth appliance of claim 5, wherein the sealing arrangement is disposed on a buccal side of the mouthpiece.

7. The mouth appliance of claim 5, wherein the sealing arrangement is disposed on a lingual side of the mouthpiece.

8. The mouth appliance of claim 5, wherein the sealing arrangement is disposed interdentally.

9. The mouth appliance of claim 5, further comprising structures configured to advance the lower jaw with respect to the upper jaw when the upper portion of the sealing arrangement is coupled to the lower portion of the sealing arrangement.

10. The mouth appliance of claim 8, further comprising structures configured to advance the lower jaw with respect to the upper jaw when the upper portion of the sealing arrangement is coupled to the lower portion of the sealing arrangement.

11. The mouth appliance of claim 5, wherein:
one or both of the upper portion and the lower portion of the sealing arrangement are movable between an engaged position and a disengaged position with respect to the upper mouthpiece and lower mouthpiece, respectively;
wherein, in the engaged position, the upper portion is coupled to the lower portion when the upper jaw and lower jaw are in the closed position; and
wherein, in the disengaged position, the upper portion is not coupled to the lower portion when the upper jaw and lower jaw are in the closed position.

12. The mouth appliance of claim 11, wherein one or both of the upper portion and the lower portion of the sealing arrangement are coupled to the corresponding mouthpiece in the engaged position and uncoupled from the corresponding mouthpiece in the disengaged position.

13. The mouth appliance of claim 11, wherein one or both of the upper portion and the lower portion of the sealing arrangement are slidable between the engaged position and the disengaged position, in relation to the corresponding mouthpiece.

14. The mouth appliance of claim 11, wherein one or both of the upper portion and the lower portion of the sealing arrangement are pivotable between the engaged position and the disengaged position, in relation to the corresponding mouthpiece.
